(19) United States
(12) Patent Application Publication
(10) Pub. No.: US 2013/0309318 A1
(43) Pub. Date: Nov. 21, 2013

(54) STABLE SILVER OXIDE FORMULATIONS

(71) Applicants: Bharat Madhavan, Kingston, RI (US); Perry Antelman, Sharon, MA (US); Shalom Lampert, Maalot (IL)

(72) Inventors: Bharat Madhavan, Kingston, RI (US); Perry Antelman, Sharon, MA (US); Shalom Lampert, Maalot (IL)

(73) Assignee: AIDANCE SKINCARE & TOPICAL SOLUTIONS LLC, Woonsocket, RI (US)

(21) Appl. No.: 13/981,393

(22) PCT Filed: Jan. 23, 2012
(86) PCT No.: PCT/US12/22220

§ 371 Date: Jul. 24, 2013

(30) Foreign Application Priority Data
Jan. 24, 2011 (GB) GB1101193.9

(51) Int. Cl. A61K 47/02; A61K 33/38

(52) U.S. Cl. CPC A61K 47/02; A61K 33/38
USPC 424/618

(57) ABSTRACT

A topical formulation for application to exposed body tissue, the formulation containing: (a) a silver oxide, and (b) at least one inorganic whitener; selected from the group of inorganic whiteners consisting of an inorganic magnesium compound and an inorganic calcium compound, said silver oxide and said inorganic whitener compound intimately dispersed within a carrier medium, and wherein a ratio of said inorganic whitener compound, to said silver oxide, is at least 0.2:1, by weight.

### CROSS-REFERENCE TO RELATED APPLICATIONS

This application draws priority from GB Patent Application No. 1101193.9, filed Jan. 24, 2011, which application is incorporated by reference for all purposes as if fully set forth herein.

### FIELD AND BACKGROUND OF THE INVENTION

The present invention relates to anti-microbial silver oxide formulations, and, more particularly, to anti-microbial silver oxide formulations having improved whiteness and stability characteristics.

Silver oxides are known to have anti-microbial properties. Silver(II) oxide is known to be more effective than silver(I) oxide.

Skin creams containing silver(II) oxide have been reported to be efficacious in treating various medical conditions, including genital herpes, oral herpes, vaginitis, vaginal yeast infections, foot and nail fungus, burns, warts, and skin infections. These skin formulations are characterized by their creaminess and ease of application, which, inter alia, enables the polyvalent silver oxide to intimately contact the skin surface.

Disadvantageously, however, the various forms of silver oxide, and silver(II) oxide in particular, are dark gray or charcoal gray powders, and are thus extremely hard to hide within white creams used in various cosmetic or pharmaceutical topical applications. Moreover, the dark silver oxide particles may stain skin and clothing.

The inventor has perceived a need for further improvements in silver oxide formulations, and the subject matter of the present disclosure and claims is aimed at fulfilling this need.

### SUMMARY OF THE INVENTION

According to the teachings of the present invention there is provided a topical formulation for application to exposed body tissue, the formulation comprising: (a) a silver oxide, and (b) at least one inorganic whitener; selected from the group of inorganic whiteners consisting of an inorganic magnesium compound and an inorganic calcium compound, said silver oxide and said inorganic whitener compound intimately dispersed within a carrier medium, and wherein a ratio of said inorganic whitener compound, to said silver oxide, is at least 0.2:1, by weight, within the formulation.

According to another aspect of the present invention there is provided a topical formulation for application to exposed body tissue, the formulation comprising: (a) a silver oxide; and (b) at least one inorganic whitener; wherein a ratio of said inorganic whitener compound, to said silver oxide, is at least 0.2:1, by weight, within the formulation.

According to further features in the described preferred embodiments, the initial whiteness value of the formulation is at least 4 reflective units.

According to still further features in the described preferred embodiments, the whiteness value of the formulation is, initially, at least 4 reflective units, at least 4.5 reflective units, at least 5 reflective units, at least 5.5 reflective units, or at least 6 reflective units, and wherein, after constant exposure to said ultraviolet light for 3 days, said value remains at least 3.5 reflective units, at least 3.75 reflective units, at least 4 reflective units, at least 4.5 reflective units, at least 5 reflective units, or at least 5.5 reflective units.

According to still further features in the described preferred embodiments, the initial whiteness value of the formulation is at least 4 reflective units, at least 4.5 reflective units, at least 5 reflective units, at least 5.5 reflective units, or at least 6 reflective units, and wherein, after constant exposure to said ultraviolet light for 3 days, a whiteness value of the formulation remains within 1.5 reflective units, within 1.25 reflective units, or within 1.0 reflective units of said initial whiteness value.

According to still further features in the described preferred embodiments, the formulation contains at least 0.05%, at least 0.10%, at least 0.2%, or at least 0.25%, by weight, of said silver oxide.

According to still further features in the described preferred embodiments, the formulation contains less than 3%, by weight, of said silver oxide.

According to still further features in the described preferred embodiments, the whitener is further selected to act as a stabilization agent that partially inhibits a darkening of the formulation when the formulation is exposed to ultraviolet light.

According to still further features in the described preferred embodiments, the formulation has a gray or light gray hue.

According to still further features in the described preferred embodiments, the carrier medium includes an oleaginous material.

According to still further features in the described preferred embodiments, the oleaginous material includes a wax.

According to still further features in the described preferred embodiments, the oleaginous material includes beeswax.

According to still further features in the described preferred embodiments, the carrier medium includes a liquid wax ester.

According to still further features in the described preferred embodiments, the liquid wax ester includes, predominantly includes, or consists essentially of jojoba oil.

According to still further features in the described preferred embodiments, the carrier medium includes a hydrogenated liquid wax ester.

According to still further features in the described preferred embodiments, the liquid wax ester includes, predominantly includes, or consists essentially of hydrogenated jojoba oil.

According to still further features in the described preferred embodiments, the whitener is an inorganic powder.

According to still further features in the described preferred embodiments, the topical formulation further comprises zinc oxide.

According to still further features in the described preferred embodiments, the whitener is further selected to act as a stabilization agent that partially inhibits a darkening of the formulation when the formulation is exposed to ultraviolet light.

According to still further features in the described preferred embodiments, the formulation contains at least 0.02%, at least 0.1%, at least 0.5%, at least 1%, at least 1.5%, at least 2%, at least 3%, at least 5%, or at least 7%, by weight, of said zinc oxide.

According to still further features in the described preferred embodiments, the formulation further includes a stabilization agent selected to partially inhibit a darkening of the formulation when exposed to ultraviolet light.

According to still further features in the described preferred embodiments, the whitener and said stabilization agent having a total concentration of at least about 0.01%, by weight, and said silver oxide having a concentration of at least about 0.01%, by weight.

According to still further features in the described preferred embodiments, the silver oxide includes, largely includes, mainly includes, predominantly includes, or consists essentially of a silver(II) oxide.

According to still further features in the described preferred embodiments, the silver oxide includes, largely includes, mainly includes, predominantly includes, or consists essentially of a silver(I) oxide.

According to still further features in the described preferred embodiments, the ratio of the stabilization agent to said zinc oxide, within the formulation, is at least 0.5:1, at least 1:1, at least 1.5:1, at least 2:1, at least 3:1, at least 5:1, or at least 7:1, by weight, said stabilization agent selected to partially inhibit a darkening of the formulation when the formulation is exposed to ultraviolet light.

According to still further features in the described preferred embodiments, the stabilization agent is selected from the group consisting of bentonite, magnesium hydroxide, calcium hydroxide, calcium carbonate, magnesium oxide, magnesium carbonate, and magnesium sulfate.

According to still further features in the described preferred embodiments, the formulation contains between 0.02% and 10% of said zinc oxide, and between 0.01% and 30% of said stabilization agent.

According to still further features in the described preferred embodiments, the inorganic whitener includes at least one inorganic whitener selected from the group consisting of magnesium oxide, magnesium hydroxide, and magnesium carbonate.

According to still further features in the described preferred embodiments, the stabilization agent includes magnesium oxide.

According to still further features in the described preferred embodiments, the formulation contains less than 0.5%, less than 0.3%, or less than 0.1% titanium dioxide, or is substantially free of said titanium dioxide.

According to still further features in the described preferred embodiments, the topical formulation further comprises zinc oxide, but contains less than 0.5%, less than 0.3%, or less than 0.1% thereof, and the ratio of said stabilization agent, said whitener, and said zinc oxide, to said silver oxide, is at least 0.5:1, at least 0.75:1, at least 1:1, at least 1.5:1, at least 2:1, at least 3:1, or at least 5:1, by weight, within the formulation.

According to still further features in the described preferred embodiments, the ratio of said stabilization agent, said whitener, and said zinc oxide, to said silver oxide, is at least 0.5:1, at least 0.75:1, at least 1:1, at least 1.5:1, at least 2:1, at least 3:1, or at least 5:1, by weight, within the formulation.

According to still further features in the described preferred embodiments, the total concentration of said whitener and said stabilization agent is at least 0.01%, at least 0.05%, at least 0.1%, at least 0.5%, at least 1%, at least 2%, at least 3%, at least 5%, at least 7%, or at least 10%.

According to still further features in the described preferred embodiments, the inorganic magnesium compound is selected from the group consisting of a magnesium oxide, a magnesium carbonate, and a magnesium sulfate.

According to still further features in the described preferred embodiments, the inorganic calcium compound is selected from the group consisting of a calcium oxide, a calcium carbonate, and a calcium sulfate.

According to still further features in the described preferred embodiments, the silver oxide has a dark hue.

According to still further features in the described preferred embodiments, the silver oxide has a hue within a range of shades between gray and black.

According to still further features in the described preferred embodiments, the formulation has a hue that is lighter than said hue of said silver oxide.

According to still further features in the described preferred embodiments, the formulation has a hue that is lighter than said hue of said silver oxide, after the formulation is subject to constant exposure to ultraviolet light for at least 3 days.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention.

In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are used to designate like elements.

In the drawings:

FIG. 1 is a graph plotting formulation whiteness, as a function of the exposure time to ultraviolet light, for formulations containing AgO (1%) and Ag₂O (1%), respectively, in a carrier base;

FIG. 1A is a graph plotting reflectance, as a function of the exposure time to ultraviolet light, for three formulations containing AgO (1%) in various carrier bases;

FIG. 2 is a graph plotting formulation whiteness, as a function of the exposure time to ultraviolet light, for the AgO formulation of FIG. 1, versus similar formulations containing AgO along with the inorganic whiteners TiO₂, and ZnO, respectively;

FIG. 3 provides a graph plotting formulation whiteness, as a function of the exposure time to ultraviolet light, for the formulations of FIG. 2, versus similar formulations containing AgO along with the inorganic substances bentonite, CaCO₃, Ca(OH)₂ and MgSO₄, respectively;

FIG. 3A presents a graph plotting normalized formulation whiteness (whiteness equals 1 at t=0), as a function of the exposure time to ultraviolet light, for the formulations of FIG. 3;

FIG. 3B is a graph plotting the absolute decrease in formulation reflectance (in RU) as a function of the exposure time to ultraviolet light, for the formulations of FIG. 3;

FIG. 4 is a graph plotting formulation whiteness, as a function of the exposure time to ultraviolet light, for the formulations of FIG. 2, versus similar formulations containing AgO along with the inorganic substances Mg(OH)₂, MgCO₃, and MgO, respectively;

FIG. 4A provides a graph plotting normalized formulation reflectance or whiteness (WN) as a function of the exposure time to ultraviolet light, for the formulations of FIG. 4;

FIG. 4B presents a graph plotting the absolute decrease in formulation reflectance as a function of the exposure time to ultraviolet light, for the formulations of FIG. 4;

FIG. 5 is a graph plotting formulation whiteness, as a function of the exposure time to ultraviolet light, showing the whiteness stabilization performance of various inorganic substances in formulations containing AgO and ZnO;

FIG. 5A is a graph comparing formulation whiteness of various formulations of FIG. 5, with the formulation whiteness of substantially identical formulations having different carrier base compositions, as a function of the exposure time to ultraviolet light;

FIG. 6 is a graph plotting the whiteness behavior of various AgO based formulations and the whiteness behavior of various Ag₂O based formulations, as a function of the exposure time to ultraviolet light;

FIG. 6A presents a graph plotting normalized formulation reflectance (WN) as a function of the exposure time to ultraviolet light, for silver oxide formulations (1% by weight) having different carrier bases;

FIG. 7 is a graph plotting formulation whiteness, as a function of the exposure time to ultraviolet light, showing the whiteness stabilization performance of various inorganic substances in formulations containing Ag₂O and ZnO;

FIGS. 8A-8C are photographs of a cloth stained with formulations of the present invention, after 3 days, 10 days, and 21 days of constant exposure to ultraviolet light;

FIG. 9 is a graph plotting formulation whiteness, as a function of the exposure time to ultraviolet light, for formulations containing AgO and varying concentrations of MgO, versus similar formulations containing solely AgO, and AgO and ZnO;

FIG. 9A presents a magnified, partial view of the graph of FIG. 9, showing exposure times of up to 3 days;

FIG. 9B provides a graph plotting normalized formulation whiteness (WN) as a function of the exposure time to ultraviolet light, for the formulations of FIG. 9A;

FIG. 10 is a bar graph showing the turbidity of a plurality of cultures, each culture containing a particular anti-microbial formulation; and

FIG. 11 is a bar graph showing the colony counts for the anti-microbial formulation containing cultures of FIG. 10.

### DESCRIPTION OF THE PREFERRED EMBODIMENTS

Before explaining at least one embodiment of the invention in detail, it is to be understood that the invention is not limited in its application to the details of construction and the arrangement of the components set forth in the following description. The invention may be capable of other embodiments or of being practiced or carried out in various ways. Also, it is to be understood that the phraseology and terminology employed herein is for the purpose of description and should not be regarded as limiting.

The medical device of the present invention contains both a silver oxide compound and a whitening agent, preferably in a carrier medium that may be a water-based cream or lotion, or an ointment that may include a wax and/or an oil. The formulation may include an emulsion, or be substantially emulsion-based.

The inventive silver oxide based medical device or formulation may have a generally white, off-white, light gray, or medium gray appearance. At lower ratios of whitening agent to silver oxide, the appearance of the medical device may be a deeper gray.

We have found that both silver(II) oxide, which is an extremely reactive material, and silver(I) oxide, which is somewhat less reactive, may deleteriously interact with various base materials within the formulation.

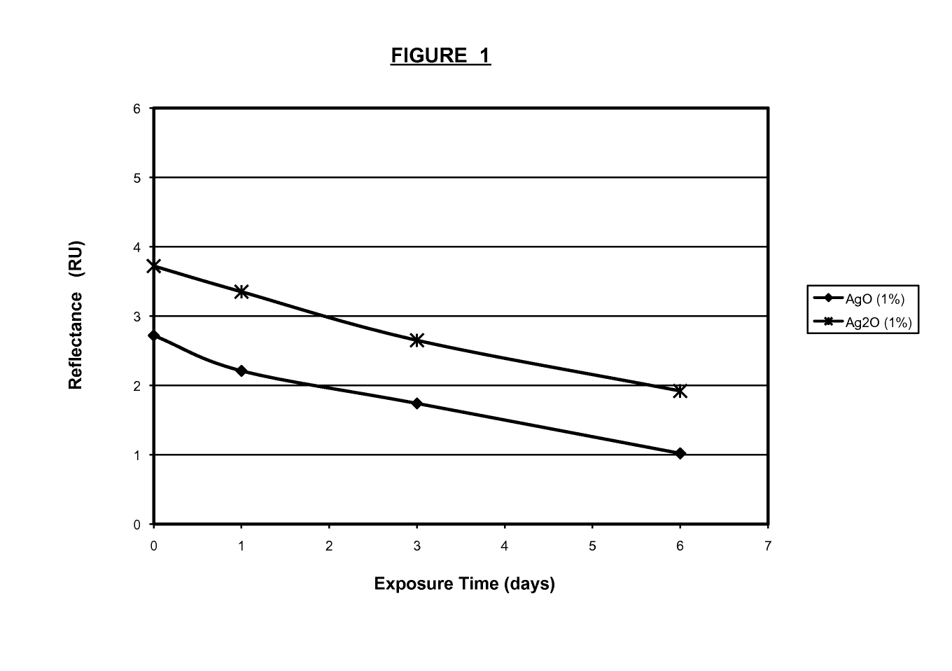

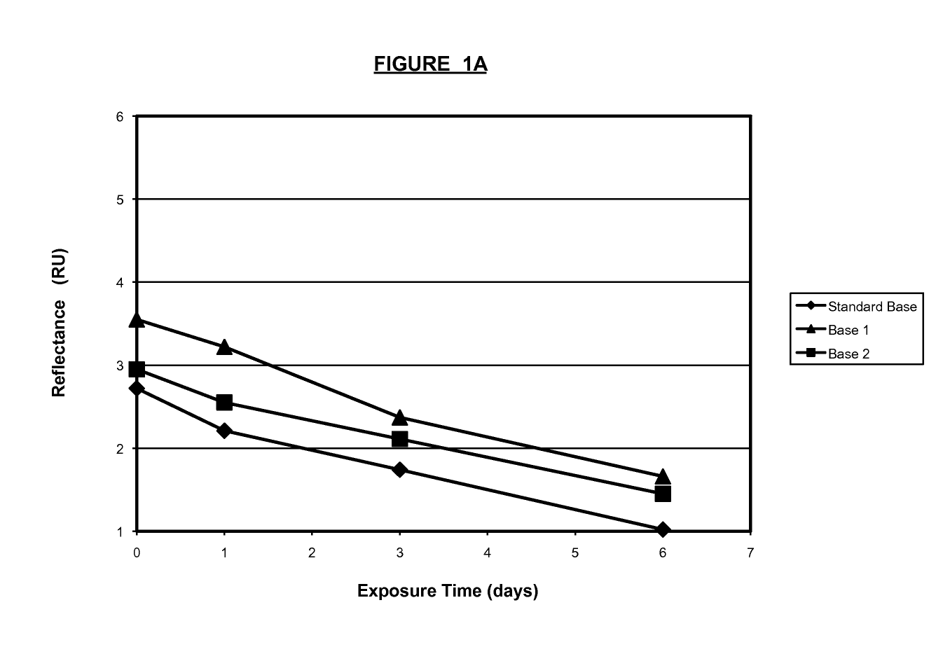

FIG. 1 is a graph plotting formulation whiteness, as a function of the exposure time to ultraviolet light, for formulations containing AgO (1%) and Ag₂O (1%), in a carrier base described in Example 1 hereinbelow. After preparing the formulations, the whiteness was measured by reflectance using a LabScan XE spectrophotometer instrument (HunterLab, VA). Initially, the AgO formulation was a relatively dark gray, and the initial value of whiteness was 2.72 reflective units. The Ag₂O formulation also had a dark gray color, albeit somewhat lighter than the AgO formulation, and exhibited an initial value of whiteness of 3.72 reflective units.

The formulations were then subjected to ultraviolet light for several days, and the whiteness of each of the formulations was monitored over time. We found that the measured reflectance or lightness of the AgO formulation decreased monotonically over time, as the formulation took on a progressively darker hue of gray. The reflectance decreased to 1.74 reflective units after 3 days, and further decreased to 1.02 reflective units after 6 days. Similarly, the measured reflectance or lightness of the Ag₂O formulation decreased monotonically over time, as the gray formulation became progressively darker. The reflectance decreased to 3.35 reflective units after 1 day, 2.65 reflective units after 3 days, and further decreased to about 1.9 reflective units after 6 days.

FIG. 1A is a graph plotting formulation whiteness, as a function of the exposure time to ultraviolet light, for three formulations containing AgO (1%): the first formulation is the formulation associated with FIG. 1, containing AgO in the “standard base” (described in detail in Example 1 hereinbelow); the second formulation contained an oxidized polyethylene homopolymer (Honeywell A-C® 629), jojoba oil, and xanthum gum (“Base 1”); and the third formulation contained beeswax, coconut oil and xanthum gum (“Base 2”). The three formulations exhibit a similar monotonous decrease in lightness over the six-day exposure period.

Without wishing to be limited by theory, we believe that silver oxide (including both silver(II) oxide and silver(I) oxide), being a reactive material, interacts with at least one other material within the formulation, causing discoloration over time. This effect may be accelerated or augmented by exposure to sunlight. In sunlight, the discoloration may be apparent even within minutes.

We have found that this effect may actually be an acceleration of a process that occurs, albeit much more slowly, when the silver oxide based formulation is packaged in a container. Thus, the discoloration phenomenon may be significant because:
topical formulations, applied to the skin, are commonly exposed to sunlight; and
the shelf life of silver oxide based formulations may be severely limited by the discoloration process occurring within the container.

By adding various whiteners during the preparation of the formulations, we found that the appearance of the formulations became significantly lighter. In the case of zinc oxide, the measured whiteness value of the silver(II) oxide formulation (1% AgO, 7% ZnO) increased by almost 3 reflectance units, to about 5.43, and the measured whiteness value of the silver(I) oxide formulation (1% Ag₂O, 7% ZnO) increased by slightly more than 3 reflectance units, to about 6.87. In the case of titanium dioxide, the measured whiteness value of the formulation (1% AgO, 7% TiO₂) nearly doubled to about 5.33. Such light-colored formulations (cream, ointment, etc.) are much more aesthetically pleasing to users, and may pose less of a problem regarding staining of skin and clothing.

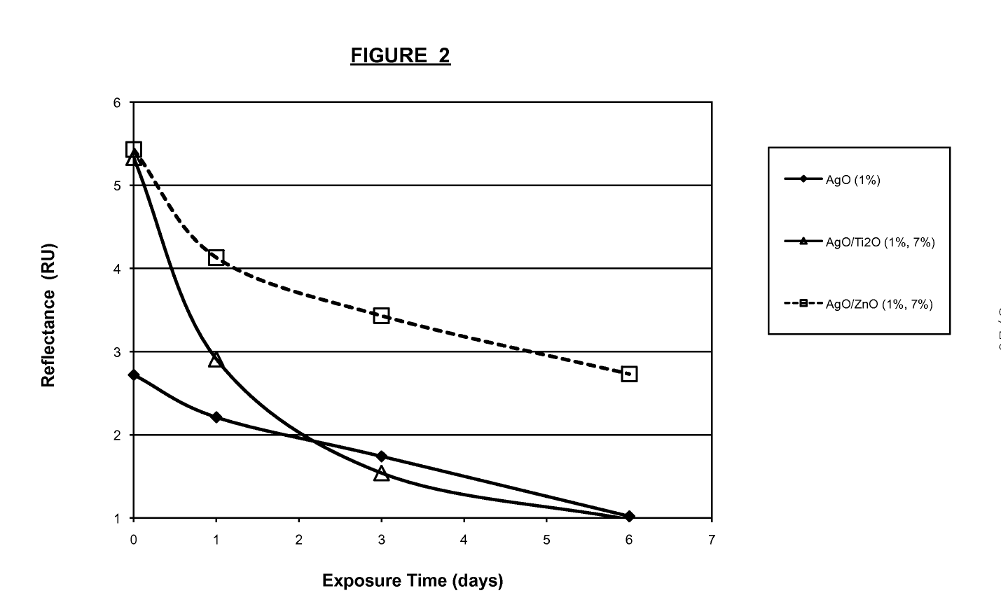

These formulations were then subjected to ultraviolet light for several days, in a procedure substantially identical to that used on the formulations described hereinabove, and the whiteness of each of the formulations was monitored over time. FIG. 2 is a graph plotting formulation lightness, as a function of the exposure time to ultraviolet light, for the AgO formulation of FIG. 1, versus similar formulations containing AgO along with the inorganic whiteners ZnO and TiO₂, respectively. It may have been expected that the whiteners would cover up a portion of the AgO, producing a lighter formulation in which the AgO is also less exposed to the ultraviolet light, such that during exposure to ultraviolet light, the decrease in whiteness might be much more moderate.

Surprisingly, we found that with both ZnO and TiO₂, the decrease in whiteness, as a function of UV exposure time, may actually be more pronounced than the corresponding decrease in whiteness of the identical formulation, without the additional whitener. In the case of the AgO/ZnO formulation, the whiteness value decreased by 2 reflective units within 3 days, and by 2.7 reflective units within 6 days.

Similar results were obtained with Ag2O: after 3 days, the whiteness value of an AgO/ZnO (1%, 7%) formulation decreased by about 1.9 reflective units; the whiteness value of an AgO/ZnO/TiO₂ (1%, 3.5%, 3.5%) formulation decreased by about 3.8 reflective units.

Thus, while the use of such whiteners greatly improved the initial formulation color, the use of these whiteners raised additional issues. Both silver(I) and silver(II) oxides may interact with the zinc oxide and titanium dioxide, or with the carrier base in the presence of zinc oxide and/or titanium dioxide, causing significant discoloration within a day or days. This effect may be accelerated or augmented by exposure to direct sunlight, in which the discoloration may be effected within minutes.

Many of the tests on AgO-based formulations have been repeated for Ag₂O-based formulations, which typically provide qualitatively similar results.

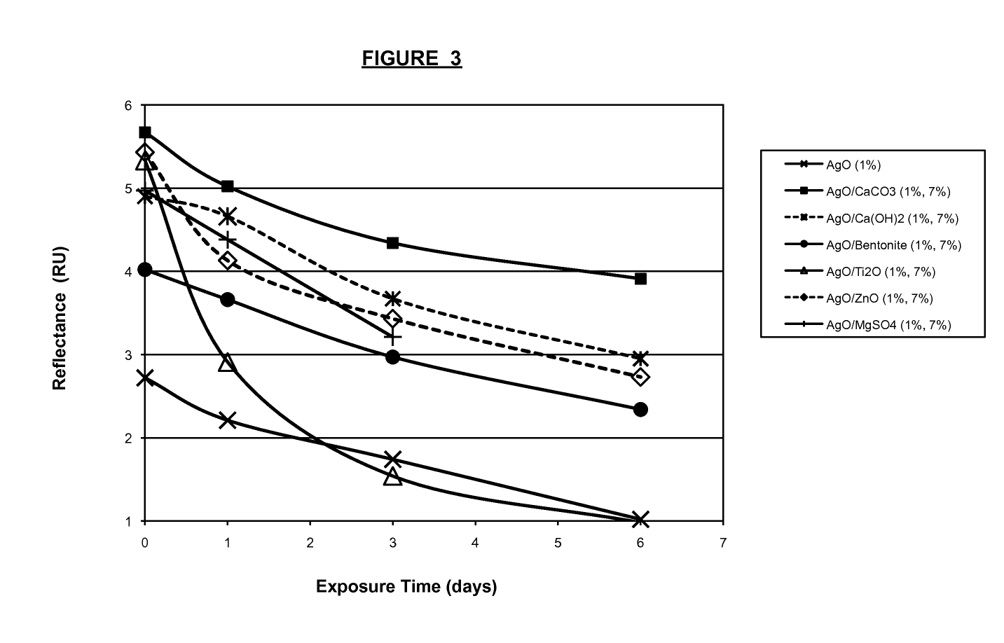

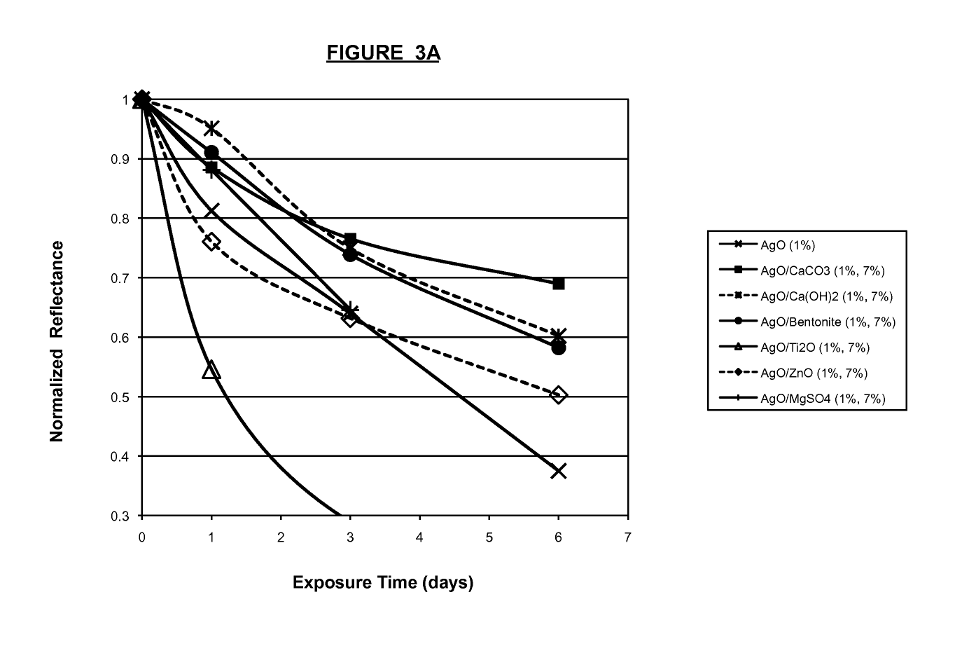

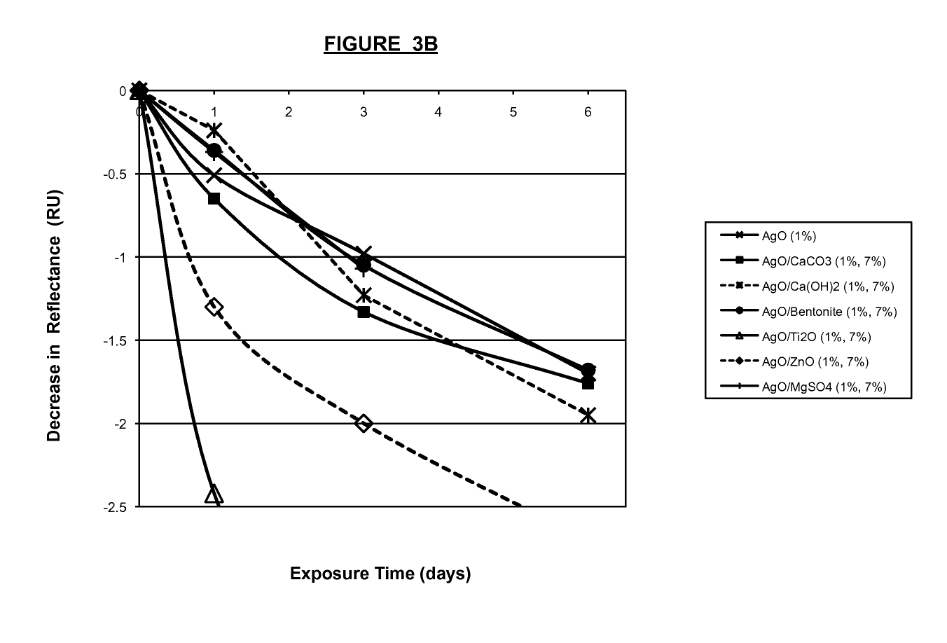

FIG. 3 is a graph plotting formulation whiteness, as a function of the exposure time to ultraviolet light, for the formulations of FIG. 2, versus similar formulations containing AgO along with the inorganic substances bentonite, CaCO₃, Ca(OH)₂ and MgSO₄, respectively. All of the mixed formulations contained 1% AgO and 7% of the additional inorganic material, to provide a firm basis of comparison.

Immediately after preparation, all of the mixed formulations whiteners were significantly lighter than the AgO standard formulation. The AgO/bentonite formulation exhibited a reflectance just over 4 reflective units, a 48% increase with respect to the AgO standard formulation. The AgO/Ca(OH)₂ and AgO/MgSO₄ formulations both exhibited a reflectance of almost 5 reflective units (4.9 and 4.97, respectively), corresponding to more than an 80% increase with respect to the AgO standard formulation. In the case of calcium carbonate, the measured whiteness value of the formulation (1% AgO, 7% CaCO₃) more than doubled to about 5.67.

The formulations were then subjected to ultraviolet light for several days, as described hereinabove, and the whiteness of each of the formulations was monitored over time. All of the formulations exhibited decreasing whiteness, as a function of UV exposure time. With the exception of the AgO/TiO₂ formulation, all of the formulations maintain a substantially higher reflectance after 1 day, after 3 days, and after 6 days. Moreover, we observe that the decreasing whiteness is less pronounced in some of the formulations.

FIG. 3A presents a graph plotting normalized formulation whiteness (W_{N}) as a function of the exposure time to ultraviolet light, for the formulations of FIG. 3. The normalization is based on the initial formulation whiteness (normalized reflectance or whiteness equals 1 at t=0). It is readily observed that the performance of the AgO/TiO₂ formulation is appreciably worse than that of the standard AgO formulation, with the normalized whiteness dropping below 0.3 after 3 days of UV exposure. The performance of the AgO/ZnO and AgO/MgSO₄ formulations is fairly similar to that of the standard AgO formulation, with the normalized whiteness dropping to about 0.63 and 0.65, respectively, after 3 days of UV exposure.

The other formulations, containing AgO along with bentonite, CaCO₃, and Ca(OH)₂, respectively, all exhibit a normalized formulation whiteness exceeding 0.7 after 3 days of UV exposure. The 1% AgO, 7% CaCO₃ formulation exhibited a normalized formulation whiteness approaching 0.7, even after 6 days of UV exposure.

FIG. 3B presents a graph plotting the absolute decrease in formulation reflectance (in RU) as a function of the exposure time to ultraviolet light, for the formulations of FIG. 3. While all of the formulations exhibited decreasing whiteness, as a function of UV exposure time, the absolute decrease in reflectance for the AgO/ZnO and the AgO/TiO₂ formulations was over 2-4 times the absolute decrease in reflectance for the AgO formulation after 1 day of UV exposure, and about 2-4 times the absolute decrease in reflectance for the AgO formulation after 3 days of UV exposure. With the exception of the AgO/TiO₂ formulation, all of the formulations maintain a substantially higher reflectance after 1 day, after 3 days, and after 6 days.

By sharp contrast, the absolute decrease in reflectance for the formulations containing AgO along with bentonite, CaCO₃, MgSO₄, and Ca(OH)₂, was reduced with respect to the absolute decrease in reflectance for the AgO formulation after both 1 day and 3 days of UV exposure.

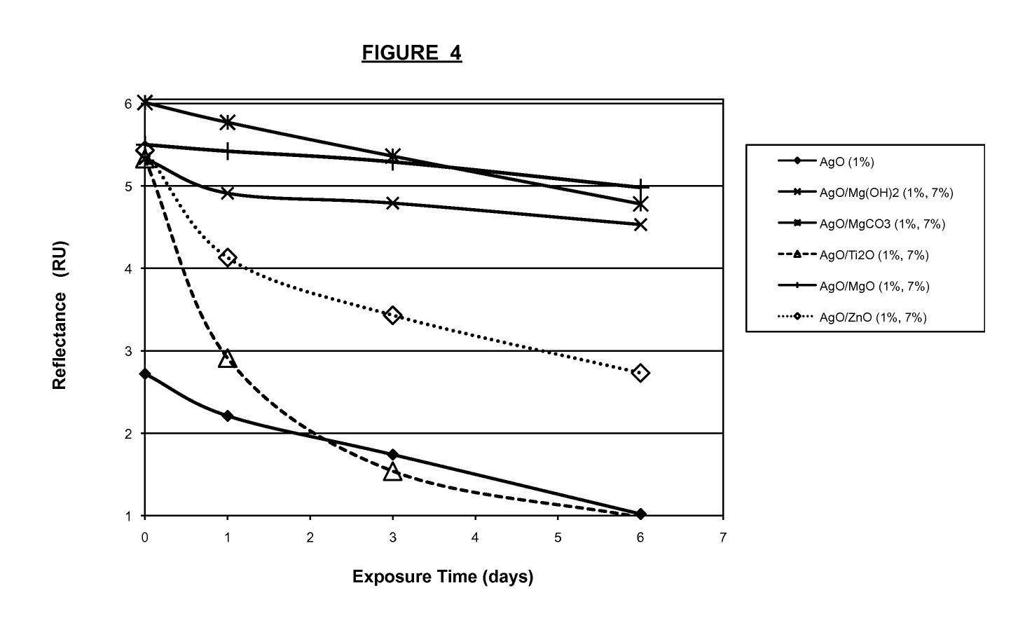

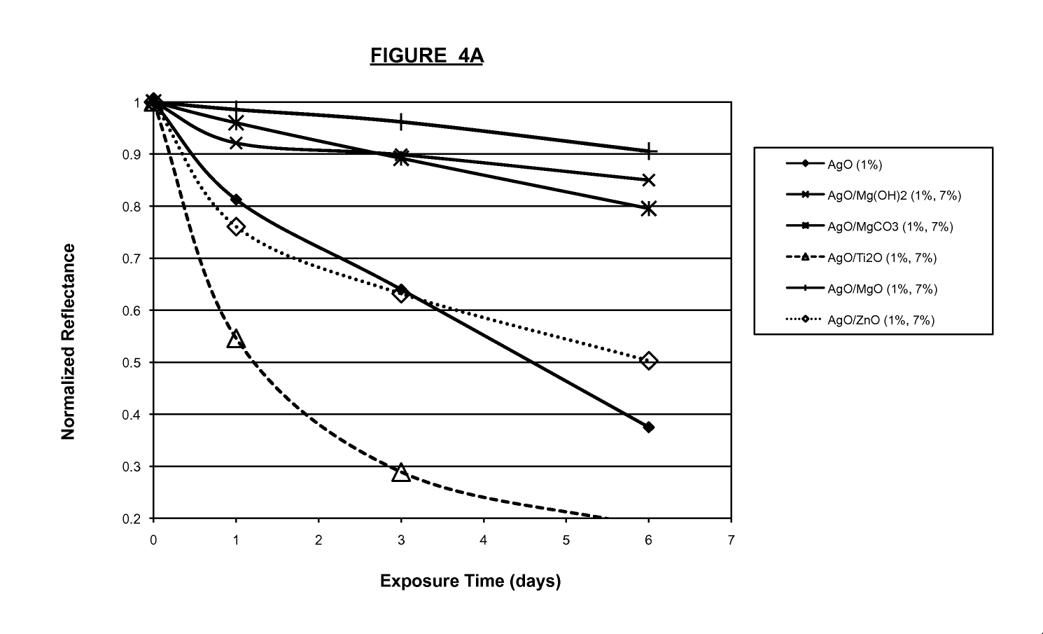

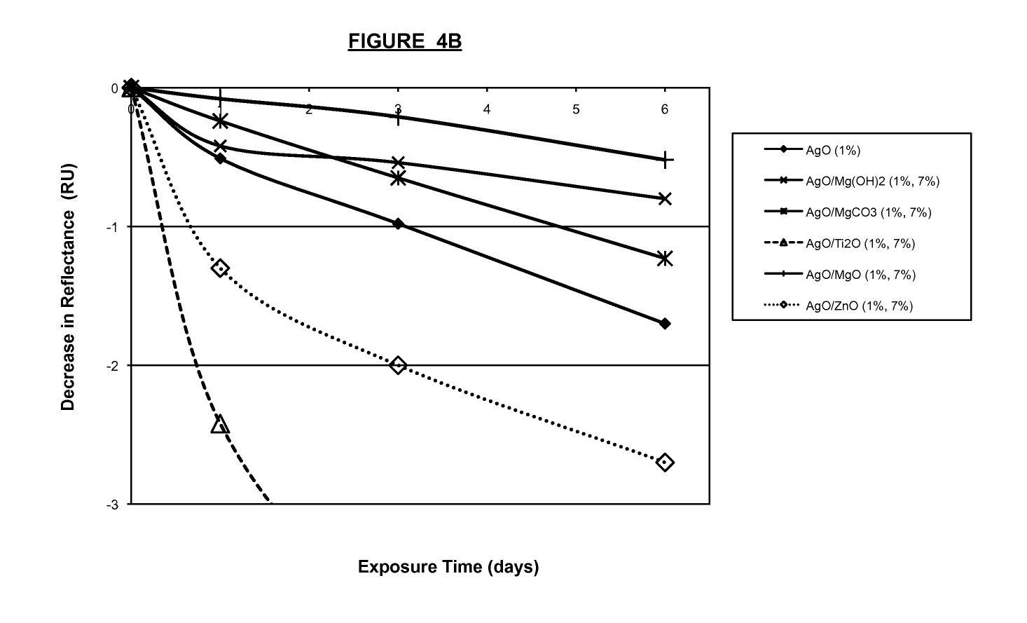

FIG. 4 is a comparison graph plotting formulation whiteness, as a function of the exposure time to ultraviolet light, for the formulations of FIG. 2, versus similar formulations containing AgO along with the inorganic substances Mg(OH)₂, MgCO₃, and MgO, respectively. All of the mixed formulations contained 1% AgO and 7% of the additional inorganic material.

Immediately after preparation, all of the mixed formulations whiteners were significantly lighter than the AgO standard formulation. The AgO/Mg(OH)₂ formulation exhibited 5.33 reflective units, a 96% increase with respect to the AgO standard formulation. The AgO/MgO and AgO/MgCO₃ formulations exhibited almost 5.5 and 6.01 reflective units, respectively, corresponding to more than a 100% or 120% increase with respect to the AgO standard formulation.

The formulations were then subjected to ultraviolet light for several days, as described hereinabove, and the whiteness of each of the formulations was monitored over time. Although these formulations exhibited decreasing whiteness, as a function of UV exposure time, the decreasing whiteness was surprisingly moderate.

FIG. 4A presents a comparison graph plotting normalized formulation whiteness (W_{N}) as a function of the exposure time to ultraviolet light, for the formulations of FIG. 4. The AgO/Mg(OH)₂, AgO/MgO and AgO/MgCO₃ formulations, all exhibit a normalized formulation whiteness approaching or exceeding 0.9 after 3 days of UV exposure, and approaching or exceeding 0.8 after 6 days of UV exposure. The 1% AgO, 7% MgO formulation exhibited a normalized formulation whiteness exceeding 0.9, even after 6 days of UV exposure.

FIG. 4B presents a graph plotting the absolute decrease in formulation reflectance (in RU) as a function of the exposure time to ultraviolet light, for the formulations of FIG. 4. While all of the formulations exhibited decreasing whiteness, as a function of UV exposure time, the absolute decrease in reflectance for the AgO/Mg(OH)₂, AgO/MgO and AgO/MgCO₃ formulations was significantly less than that of the AgO formulation after 1 day of UV exposure, after 3 days of UV exposure, and after 6 days of UV exposure.

Over the course of clinical trials, we have found that treating wounds with an AgO/ZnO formulation may be considerably more efficacious than conventional treatments used in the controls. Moreover, the AgO/ZnO formulation exhibited a higher efficacy than a similar formulation containing a comparable concentration of AgO, but no ZnO. In addition, the AgO/ZnO formulation was found to improve the microcirculation and healing rate in both venous ulcerations and diabetic ulcerations.

The darkening of the AgO/ZnO formulation over time, and during exposure to UV light, may be a disadvantage in many applications. As best seen in FIG. 2, the darkening of AgO/ZnO formulations may be rapid and appreciable. After only one day of UV exposure, the “white” AgO/ZnO formulation has become nearly as dark as the identical formulation, without ZnO; after three days of UV exposure, the AgO/ZnO formulation looks extremely similar to that formulation, and may actually be even darker. Thus, while the ZnO contributes to the formulation efficacy, the contribution to the whiteness may be surprisingly modest. In addition, the dark appearance of the formulation, developed over time, may reduce patient compliance.

We have found that the appearance of formulations containing AgO and ZnO may be greatly enhanced by the addition of at least one stabilization agent adapted to at least partially inhibit a darkening of the formulation when exposed to ultraviolet light. The stabilization agent may advantageously act as a whitener as well.

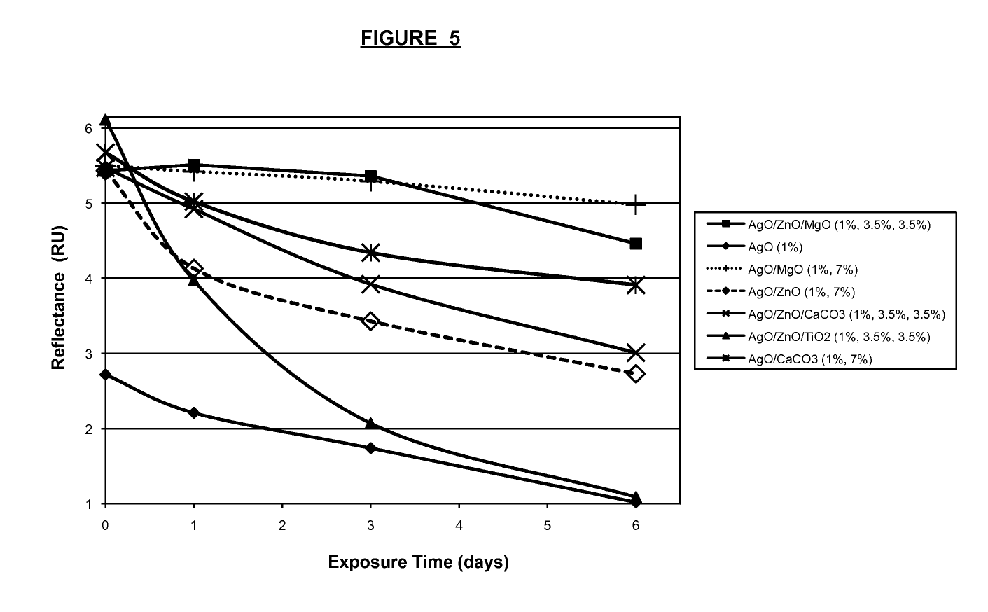

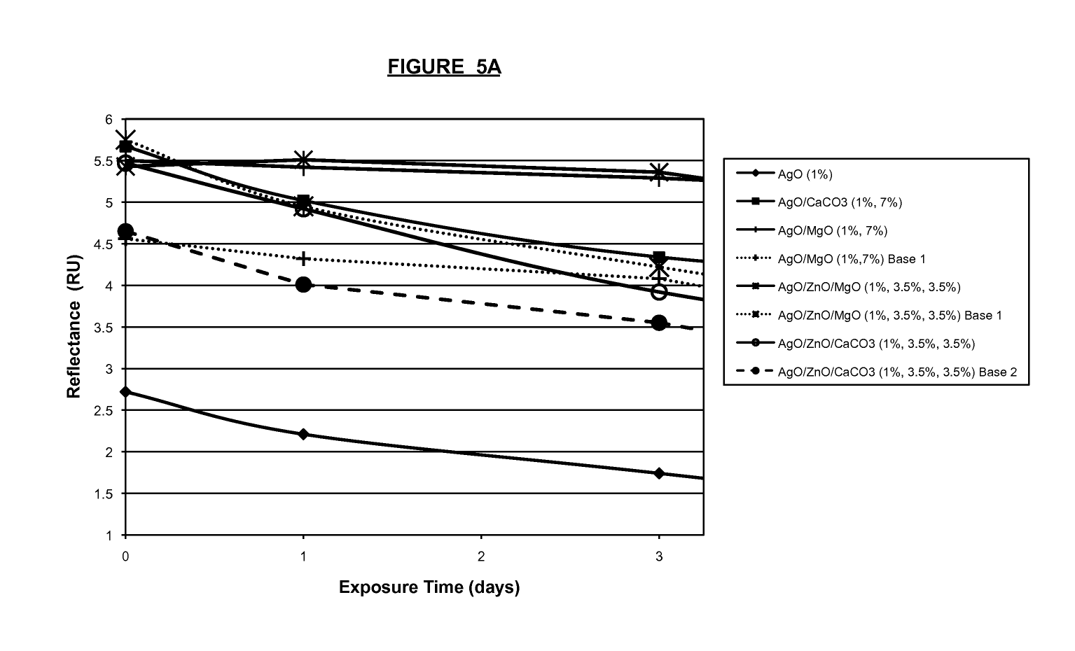

FIG. 5 is a graph plotting formulation whiteness, as a function of the exposure time to ultraviolet light, showing the whiteness stabilization performance of various inorganic substances in formulations containing AgO and ZnO. As described hereinabove, the decrease in whiteness of the AgO/ZnO control or base formulation (1% AgO/7% ZnO), as a function of UV exposure time, is more pronounced than the corresponding decrease in whiteness of the identical AgO formulation, without the zinc oxide. When half of the zinc oxide is replaced with titanium dioxide, the initial whiteness is improved, but after three days of UV exposure, the whiteness exhibited by the AgO/ZnO/TiO₂ formulation (1% AgO/3.5% ZnO/3.5% TiO₂) is significantly lower than that of the base formulation.

By sharp contrast, when half of the zinc oxide is replaced with magnesium oxide, the initial whiteness is substantially maintained, but over the course of several days of UV exposure, the whiteness exhibited by the AgO/ZnO/MgO formulation (1% AgO/3.5% ZnO/3.5% MgO) is significantly higher than that of the base formulation. Surprisingly, the whiteness stays fairly constant over the first three days of UV exposure, and—perhaps even more surprisingly, the whiteness exhibited appears to be very similar to the whiteness (plotted in FIG. 5, as a reference line) of an AgO/MgO formulation containing 1% AgO, 7% MgO, and having no ZnO.

Similarly, when half of the zinc oxide is replaced with calcium carbonate (CaCO₃), the initial whiteness is substantially maintained. Over the course of several days of UV exposure, the whiteness exhibited by the AgO/ZnO/CaCO₃ formulation (1% AgO/3.5% ZnO/3.5% CaCO₃) is somewhat higher than that of the base formulation. Surprisingly, through the third day of UV exposure, the whiteness exhibited appears to be similar to the whiteness (plotted in the Figure, as a reference line) of an AgO/CaCO₃ formulation containing 1% AgO, 7% CaCO₃, and having no ZnO.

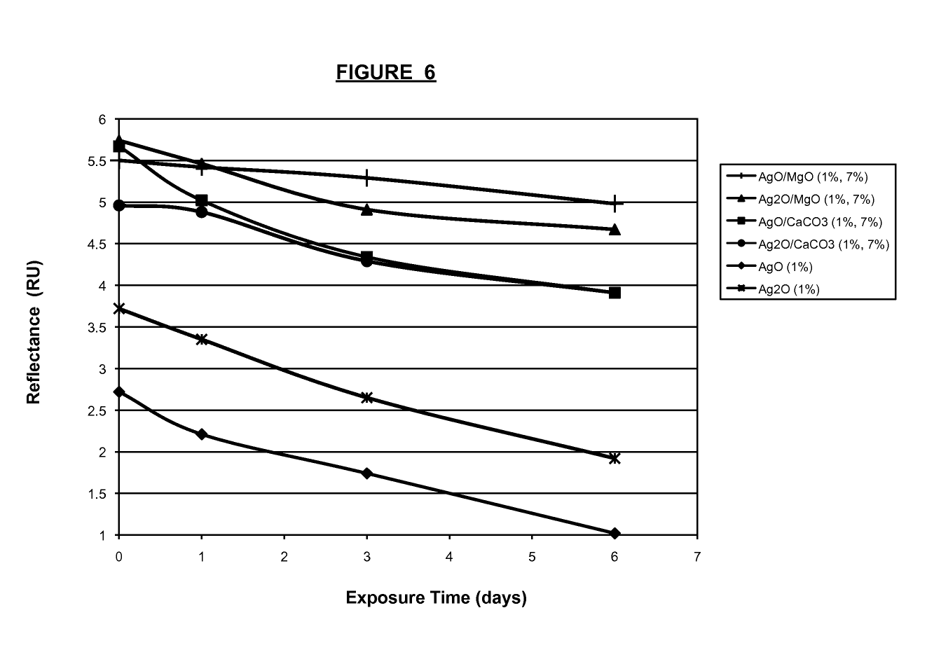

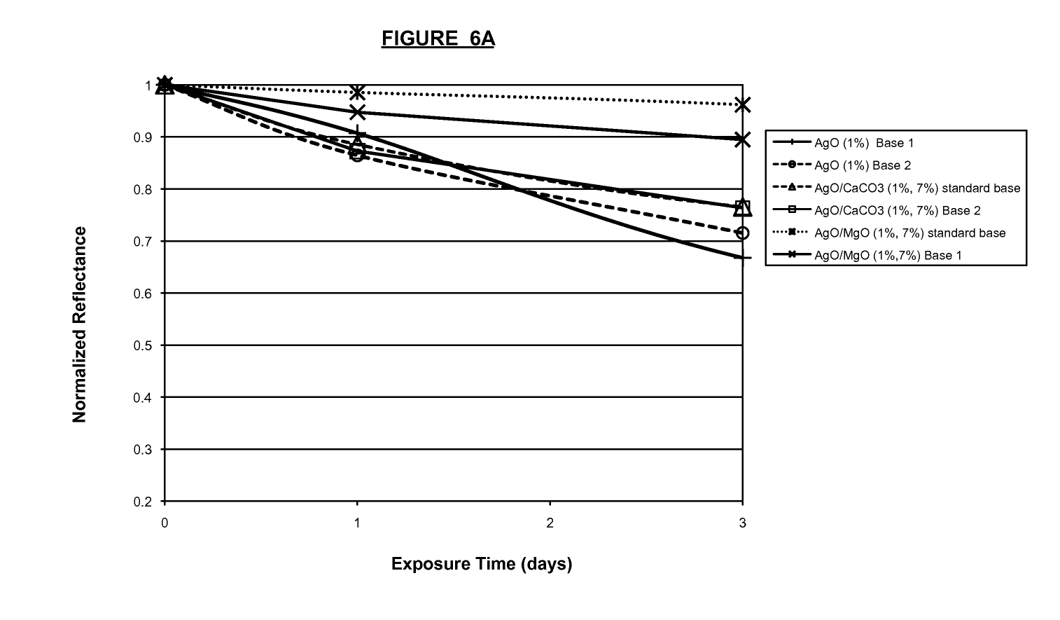

FIG. 6 is a graph plotting the whiteness behavior of various AgO based formulations and the whiteness behavior of various Ag₂O based formulations, as a function of the exposure time to ultraviolet light. All of the mixed formulations contained 1% AgO or Ag₂O, and 7% of the additional inorganic material—MgO or CaCO₃, respectively.

The formulations were subjected to ultraviolet light for several days, as described hereinabove, and the whiteness of each of the formulations was monitored over time. Although the mixed formulations exhibited decreasing whiteness as a function of UV exposure time, the decreasing whiteness was surprisingly moderate for both AgO-based and Ag₂O-based mixed formulations.

Moreover, we have found with these exemplary mixed formulations, as well as with other mixed formulations, that the behavior of the Ag₂O-based formulations and the Ag₂O-based formulations, with respect to UV light exposure, is strikingly similar. By way of example, the measured whiteness for AgO/CaCO₃, and Ag₂O/CaCO₃, is virtually identical for exposure times of 1 day, 3 days and 6 days.

Similarly, the measured whiteness values for the AgO/MgO and Ag₂O/MgO formulations, respectively, are within 5% of each other initially, and remain within about 7% of each other for exposure times of 1 day, 3 days and 6 days.

FIG. 6A presents a graph plotting normalized formulation reflectance or whiteness (W_{N}) as a function of the exposure time to ultraviolet light, for six silver oxide formulations (1% by weight) having different carrier bases. Two of the formulations contained solely (i.e., sans whiteners or stabilization agents) 1% AgO in Base 1 or in Base 2, respectively; two of the formulations contained 1% AgO and 7% CaCO₃, in the standard base or in Base 2, respectively; and two of the formulations contained 1% AgO and 7% MgO in the standard base or in Base 1, respectively.

Despite the significant differences in the chemical and physical properties of the carrier bases, we observe that the behavior of each type of formulation appears to be largely insensitive to the composition of the carrier base.

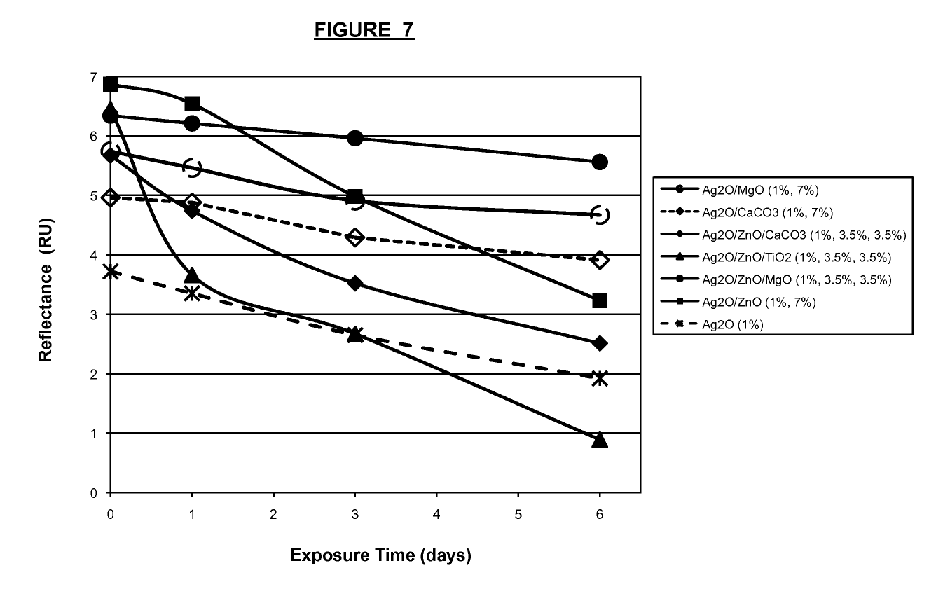

FIG. 7 provides a graph plotting formulation whiteness, as a function of the exposure time to ultraviolet light; the graph demonstrates the whiteness stabilization performance of various inorganic substances in formulations containing Ag₂O and ZnO. The decrease in whiteness of the Ag₂O/ZnO control or base formulation (1% Ag₂O/7% ZnO), as a function of UV exposure time, may be more pronounced than the corresponding decrease in whiteness of the identical Ag₂O formulation, without the zinc oxide (not shown). When half of the zinc oxide is replaced with titanium dioxide, the results are still very poor: after only one day of UV exposure, the whiteness value exhibited by the Ag₂O/ZnO/TiO₂ formulation (1% Ag₂O/3.5% ZnO/3.5% TiO₂) is more than 40% lower than the initial whiteness.

By sharp contrast, when half of the zinc oxide is replaced with magnesium oxide, the initial whiteness value is somewhat lower, but after three days of UV exposure, the whiteness value exhibited by the Ag₂O/ZnO/MgO formulation (1% Ag₂O/3.5% ZnO/3.5% MgO) is significantly higher than that of the Ag₂O—ZnO formulation (1% Ag₂O/7% ZnO). Surprisingly, the whiteness value stays fairly constant over the first three days of UV exposure. The whiteness value exhibited appears to be higher than the whiteness value (plotted in FIG. 7 as a reference line) of an Ag₂O/MgO formulation containing 1% Ag₂O, 7% MgO, and having no ZnO.

The Ag₂O/ZnO/CaCO₃ formulation (1% AgO/3.5% ZnO/3.5% CaCO₃) exhibits poor whiteness values when compared with both 1% Ag₂O/7% ZnO and with 1% Ag₂O/7% CaCO₃.

Typically, the inventive formulations contain up to 5% silver oxide or up to 3% silver oxide, by weight. More typically, the formulations contain 0.01% to 3% silver oxide. The ratio of the whitener (without zinc oxide) and the stabilization agent to the silver oxide, within the formulation, is typically at least 0.2:1, at least 0.3:1, at least 0.5:1, at least 1:1, at least 2:1, at least 3:1, at least 4:1, at least 5:1, at least 7:1, or at least 10:1, by weight.

In preparing the various formulations of the present invention, we have discovered that within a specified range of weight ratios and/or compositions, the silver oxide based formulation is highly spreadable, despite the presence of the chalky whiteners and/or stabilization agents. We have found that formulations containing more than 20-25%, by weight, of the whiteners and/or stabilization agents, may display poor spreadability, and may generally be less efficacious from an anti-microbial standpoint.

Thus, the inventive formulations may contain up to 20% by weight, of at least one whitener and/or stabilization agent, more typically, up to 17% by weight, and more typically, up to 15% by weight. The formulations may typically contain at least 0.2%, at least 0.5%, at least 1%, at least 2%, at least 3%, or at least 5%, by weight, of the whitener and/or stabilization agent. Most formulations contain between 2% and 15%, between 2.5% and 12%, or between 3% and 10%, by weight, of the whitener and/or stabilization agent.

When both a whitener (e.g., zinc oxide) and a stabilization agent (e.g., Mg(OH)₂ or MgO) are used, the formulations may typically contain at least 0.2%, at least 0.5%, at least 0.8%, at least 1%, at least 2%, at least 3%, or at least 5%, by weight, of the whitener, and more typically, between 0.8% to 10%, between 0.8% to 8%, or between 0.8% to 6%, by weight. The formulations may typically contain at least 0.2%, at least 0.5%, at least 0.8%, at least 1%, at least 2%, at least 3%, or at least 5%, by weight, of the stabilization agent, and more typically, between 0.8% to 8%, between 0.8% to 6%, or between 0.8% to 4%.

The ratio of stabilization agent to whitener may vary greatly, but is typically at least 0.1:1, at least 0.25:1, at least 0.5:1, at least 1:1, at least 2:1, at least 3:1, or at least 5:1, by weight. Typically, the ratio of stabilization agent to whitener may be up to 15:1, up to 12:1, up to 10:1, or up to 8:1, by weight.

### EXAMPLES

Reference is now made to the following examples, which together with the above description, illustrate the invention in a non-limiting fashion.

### Example 1

The exemplary “standard base” silver oxide formulations provided hereinabove were prepared according to the following general procedure: jojoba oil was heated to 80° C. Beeswax was introduced, and the material was mixed thoroughly during cooling to about 55° C. Palmarosa oil was added, followed by at least one of a silver (II) oxide (AgO) and silver (I) oxide (Ag₂O). Where appropriate, a solid (acting as a whitener and/or stabilization agent) such as an inorganic powder was introduced along with the silver oxide, although practical considerations may suggest that the addition be made prior to the introduction of the silver oxide, or sometime thereafter.

Mixing may be maintained throughout, and during cooling of the mixture to 35° C.-40° C.

Alternatively or additionally, various base components that are known to those skilled in the art may be used, including petrolatum, polyethylene polymers (such as: oxidized polyethylene homopolymer (Honeywell A-C® 629)), mineral oil, coconut oil, xanthum gum.

In the exemplary formulations described hereinabove, the weight ratio of the jojoba oil to beeswax was about 5.5 to 1. The palmarosa oil content was about 0.04% of the jojoba oil content. The total content of the beeswax, jojoba oil, silver oxide, and one or more whiteners and stabilization agents, within the formulations, typically exceeded 99%.

### Example 2

An exemplary general procedure for producing the inventive silver oxide based cream is as follows: a base material such as liquid wax ester (e.g., jojoba oil) is heated, preferably to around 80° C. Alternatively or additionally, various base components may be used, base components that will be known to those skilled in the art of topical formulation production, such as, but not limited to, petrolatum, polyethylene polymers (such as an oxidized polyethylene homopolymer (Honeywell A-C® 629)), mineral oils, coconut oil, and xanthum gum.

A thick base material (e.g., a wax such as beeswax, polyethylene polymers, or hydrogenated jojoba oil or the like) may be melted into the liquid wax ester or base material. The mixture may be mixed thoroughly as it is cooled, typically below about 60° C. An essential oil such as palmarosa oil may be added. Mixing may be continued as at least one of a whitener and a stabilization agent (both in the form of solid powders) is introduced. At least one silver oxide such as a silver (II) oxide or a silver (I) oxide is also introduced, before, after, or concurrently with the whitener and stabilization agent, and the mixing may be continued during cooling of the mixture to below about 40° C. The mixing may advantageously produce an intimately dispersed formulation in which the silver oxide and the whitener and/or stabilization agent may be distributed in a homogeneous or substantially homogeneous fashion within the carrier medium.

### Example 2A

Water-based and emulsion-based formulations according to the present invention may be prepared according to the following exemplary procedure: to a container containing water or an aqueous solution may be added a viscosity-building agent (e.g., a smectite such as a bentonite or montmorillonite powder such as Gelwhite H, produced by Southern Clay Products, Inc., Gonzales, Tex.). Other viscosity-building clays, particularly clays in which the silicate layers are disposed in a sandwiched structure, may also be used. Other viscosity-building agents and thickeners may be used, e.g., carbomers. Preferably, such selected materials may exhibit good resistance to oxidation or chemical attack by the silver oxide or oxides.

The mixture is mixed or homogenized, typically for 0.5 to 2 hours. Silver(II) oxide and/or silver(I) oxide may be introduced at this stage of the processing. The whitener(s) and/or stabilization agent(s) may be introduced to the mixture, typically along with the silver oxide, or sometime therebefore or thereafter. The oil and/or liquid wax ester (e.g., jojoba oil) may be introduced to the mixture during the mixing (e.g., blending or homogenizing).

### Examples 3-8

Six formulations were prepared according to the general procedure provided above. The control formulation contained 1% AgO, 7% ZnO, and no additional whitener or stabilizing agent. The other five formulations contained various quantities of MgO, such that the total amount of zinc oxide and magnesium oxide equaled 7%. The composition of each formulation, along with the weight ratios of magnesium oxide to zinc oxide and magnesium oxide to silver oxide, are provided below in TABLE 1:

TABLE 1

| | COMPOSITION (Wt. %) | | | WEIGHT RATIOS | |
|---|---|---|---|---|---|
| Formulation | AgO | ZnO | MgO | MgO:ZnO | MgO:AgO |
| A (Example 3) | 1 | 7 | 0 | 0.00 | 0.00 |
| B (Example 4) | 1 | 6.3 | 0.7 | 0.11 | 0.70 |
| C (Example 5) | 1 | 4.9 | 2.1 | 0.43 | 2.10 |
| D (Example 6) | 1 | 3.5 | 3.5 | 1.00 | 3.50 |
| E (Example 7) | 1 | 2.1 | 4.9 | 2.33 | 4.90 |
| F (Example 8) | 1 | 0.7 | 6.3 | 9.00 | 6.30 |

About 1 gram of each of Formulations B-F was smeared on a piece of 100% white cotton cloth, which was then exposed to white UV light for about one month. Periodically, the cloth was examined for change in color, and photographed.

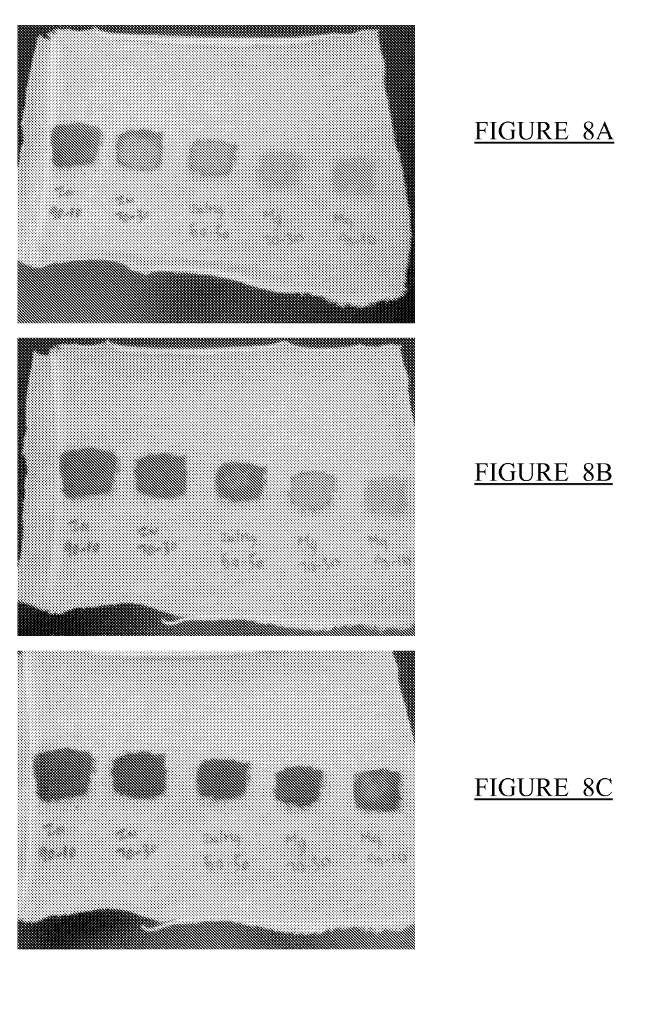

FIG. 8A provides a photograph of Formulations B-F (ordered from left to right) after 3 days of constant exposure to UV light. It is clearly observed that staining or darkness is inversely related to the amount of MgO. Formulation B, though having a rather dark appearance, is actually lighter than Formulation A (not shown). The stain from Formulation C is dark solely near the perimeter; the stain associated with Formulation D is dark solely near a portion of the perimeter; the stains associated with Formulations D and E, respectively, are light throughout. Thus, as the content of MgO within the formulation becomes correspondingly higher, the staining appears to be lighter and less pronounced. It is further evident from the stains and from the data in TABLE 1 that reduced staining (or formulation lightness) may be proportionally related to, or may positively depend on, at least one of the weight ratio of MgO:ZnO and MgO:AgO.

These observations may be further supported by the photographs of the stains provided in FIGS. 8A and 8B, taken 10 days and 21 days, respectively into the experiment. As before, the cloth underwent constant exposure to UV light. After 10 days, Formulation D is lighter than Formulation B after 3 days. Even after 21 days, Formulation F is lighter than Formulation B after 3 days.

Perhaps more significantly, all of the formulations containing MgO appear to be lighter than corresponding Formulation A, which contains no MgO.

### Example 9

Formulation reflectance, lightness, or whiteness was evaluated as follows: approximately 1 gram of a particular sample (typically an ointment or cream) was spread on a 5 cm by 5 cm area of white cotton cloth and distributed evenly, typically using a metal spatula.

A LabScan XE spectrophotometer instrument (HunterLab, VA) was used to evaluate the reflectance of each sample. The working principle of the instrument pertains to the property of light reflection. The cloth sample is stored in a completely dark container. To measure the reflectance, the instrument exposes the sample to a controlled, repeatable pulse of light. The lightness of the sample is generally correlated with the reflectance: higher values correspond to lighter samples.

The spectrophotometer has a wavelength range of 375 nm to 750 nm and an optical resolution of 10 nm. The spectrophotometer measures reflected color using 0°/45° geometry.

### Example 10

Formulation reflectance was evaluated as a function of the exposure time to ultraviolet light, as follows: the LabScan XE spectrophotometer described in Example 9 was used. Each sample was continuously exposed to ultraviolet light produced by the illumination source. The continuous UV exposure is through a 254 nm, 6W UV bulb distributed by Cole-Parmer®. The distance between the UV source and the specimen or formulation was 18 inches (˜45.7 cm).

Sample preparation was substantially the same as that described in Example 9. After an initial measurement (“day 0”), additional measurements were made over the course of the exposure to ultraviolet light, typically on days 1, 3 and 6.

### Examples 11-15

Five formulations were prepared according to the general procedure provided above. All the formulations contained 1% AgO, and were distinguished by their varying concentrations of MgO. Example 3 is provided for comparative purposes. The composition of each formulation, along with the weight ratios of magnesium oxide to silver oxide, are provided below in TABLE 2:

TABLE 2

| | COMPOSITION (Wt. %) | | | WEIGHT RATIO |
|---|---|---|---|---|
| Formulation | AgO | ZnO | MgO | MgO:AgO |
| G (Example 3) | 1 | 7 | 0 | 0.0 |
| H (Example 11) | 1 | 0 | 0 | 0.0 |
| I (Example 12) | 1 | 0 | 3.5 | 3.5 |
| J (Example 13) | 1 | 0 | 7.0 | 7.0 |
| K (Example 14) | 1 | 0 | 14 | 14 |
| L (Example 15) | 1 | 0 | 28 | 28 |

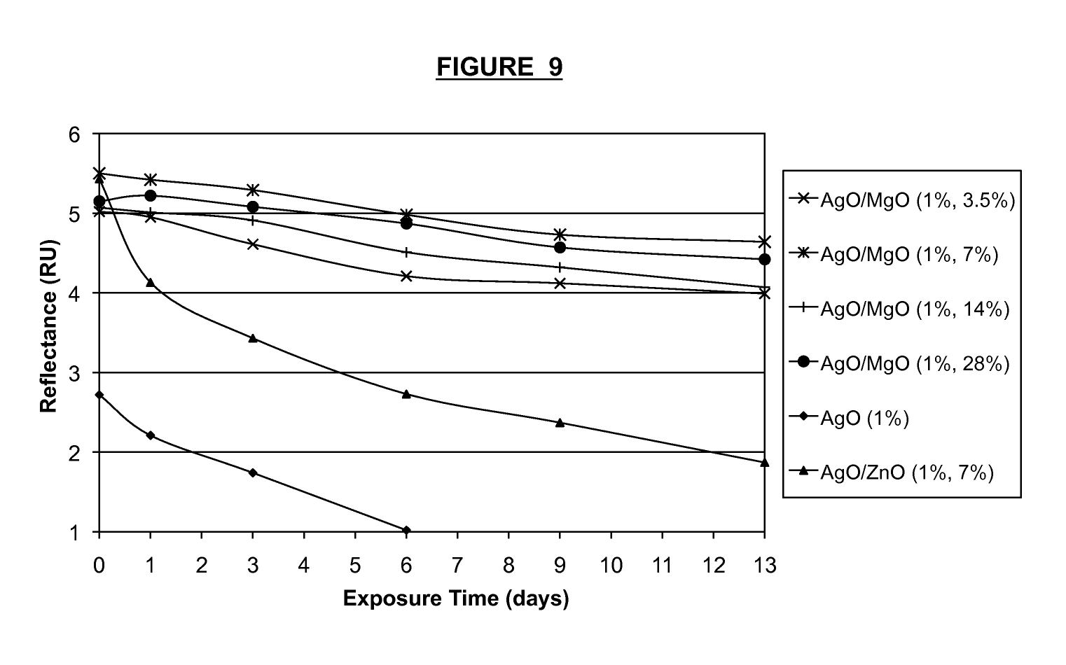

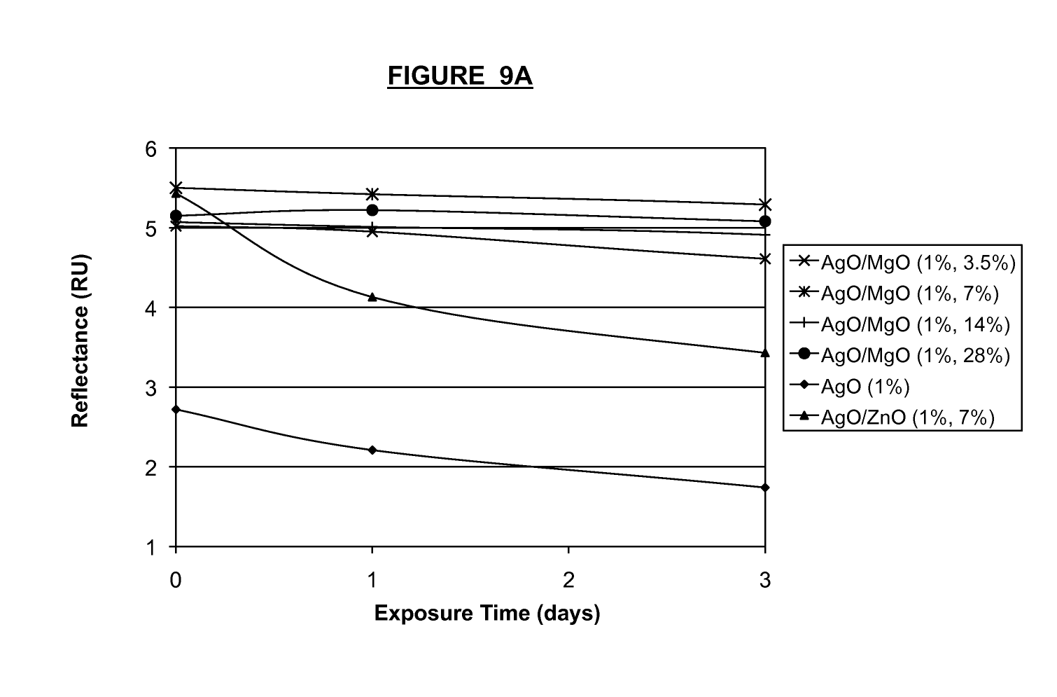

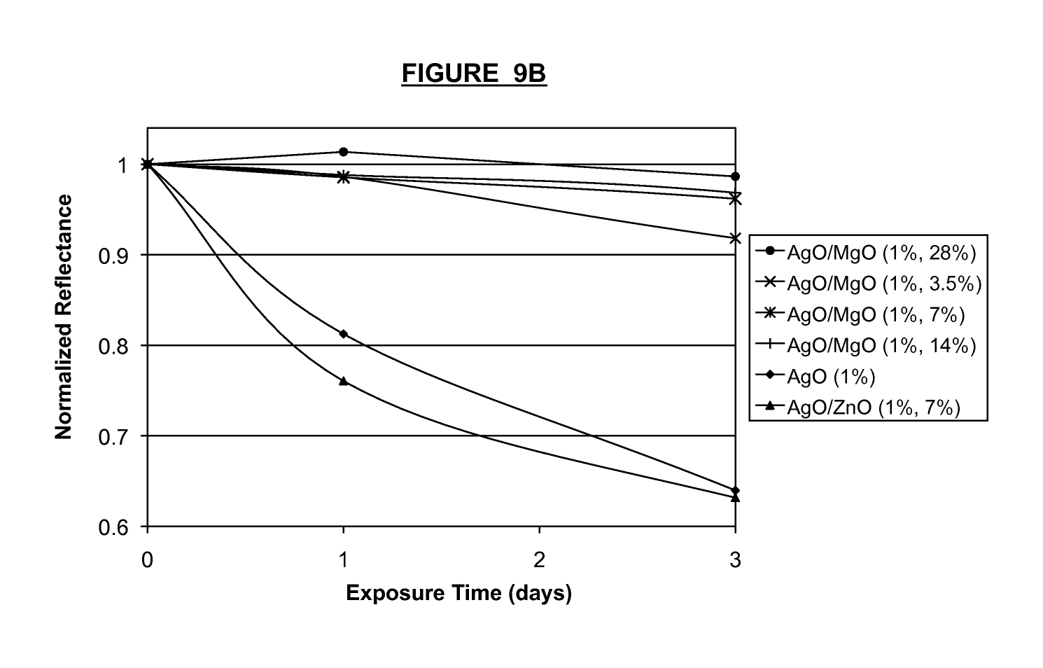

FIG. 9 is a graph plotting formulation whiteness, as a function of the exposure time to ultraviolet light, for formulations containing AgO and varying concentrations of MgO, versus similar formulations containing solely AgO (Example 11), and AgO and ZnO (Example 3). FIG. 9A is a magnified, partial view of the graph of FIG. 9, showing exposure times of up to 3 days. FIG. 9B provides a graph plotting normalized formulation whiteness (W_{N}) as a function of the exposure time to ultraviolet light, for the formulations of FIG. 9A.

We observe that in all of the formulations containing MgO, the MgO behaved as a whitener and as a stabilizing agent. The initial whiteness values of the MgO-containing formulations were about 5 to 5.5 reflectance units. After one day of exposure to ultraviolet light, the whiteness values of the MgO-containing formulations dropped slightly, remaining close to or about 5 to 5.5 reflectance units. After three days of exposure to ultraviolet light, the whiteness values of the MgO-containing formulations dropped slightly, to about 4.6 to 5.3 reflectance units. After thirteen days of exposure to ultraviolet light, the whiteness values of the MgO-containing formulations dropped slightly, to about 4.0 to 4.6 reflectance units.

In FIG. 9B, we observe that after three days of exposure to ultraviolet light, the Mg0-based formulations retained between 92% and 98% of their initial whiteness values. Formulation L, containing a 28:1 ratio of MgO to AgO, retained approximately 98% of its initial whiteness value; Formulation K, containing a 14:1 ratio of MgO to AgO, retained approximately 97% of its initial whiteness value; Formulation J, containing a 7:1 ratio of MgO to AgO, retained approximately 96% of its initial whiteness value; and Formulation I, containing a 3.5:1 ratio of MgO to AgO, retained approximately 92% of its initial whiteness value. Another formulation, containing a 1:1 ratio of MgO to AgO, retained almost 80% of its initial whiteness value, after three days of exposure to ultraviolet light. Moreover, even a formulation containing a 0.5:1 ratio of MgO to AgO acted as a stabilization agent over the course of at least one day of exposure to ultraviolet light.

All of these results are exceptionally good when compared with Formulation G, containing a 7:1 ratio of ZnO to AgO, which retained only about 63% of its initial whiteness value, after three days of exposure to ultraviolet light.

### Example 16

Silver oxide formulations were prepared according to the general procedure provided in Example 2A. The active ingredients were:
silver(II) oxide (0.05% to 1.5%);
zinc oxide (1% to 11.3%);
at least one additional whitener or stabilization agent (1% to 9% magnesium hydroxide, calcium hydroxide, bentonite, calcium carbonate, magnesium oxide, magnesium carbonate, or magnesium sulfate).

Carrier ingredients were selected from beeswax, benzoic acid, bentonite, dimethicone, glycine, soybean oil, methylparaben, microcrystalline wax, mineral oil, panthenol, propylene glycol, propylparaben, sodium hydroxide, sorbitan sesquioleate, tocopheryl acetate, and water. A minute amount of fragrance was added to some of the formulations.

The silver(II) oxide based formulations generally exhibited an off-white or light gray appearance, suitable for topical formulations.

### Example 17

Silver oxide formulations were prepared according to the general procedure provided in Example 2A. The active ingredients were:
silver(I) oxide (at least 0.1% to 3%);
zinc oxide (1% to 8%);
at least one additional whitener or stabilization agent (1% to 9% magnesium hydroxide, calcium hydroxide, bentonite, calcium carbonate, magnesium oxide, magnesium carbonate, or magnesium sulfate).

The carrier ingredients were substantially the same as those used in Example 16.

The silver(I) oxide based formulations generally exhibited an off-white, light gray, or medium gray appearance, suitable for topical formulations.

### Example 18

Silver oxide formulations were prepared according to the general procedure provided in Example 1. The active ingredients were:
silver(I) oxide or silver(II) oxide (0.05%);
zinc oxide (0.0375%);
magnesium oxide (0.01%; 0.0375%);

The carrier was the standard base described in Example 1. The ratio of whitener and stabilization agent to silver oxide was 0.2:1 and 0.75:1. The ratio of whitener (and stabilization agent to zinc oxide was 0.27:1 and 1:1. The ratio of total whitener, zinc oxide and stabilization agent to silver oxide was 0.95:1 and 1.5:1. Under these conditions, both the silver(I) and the silver(II) oxide based formulations exhibited a generally off-white to slightly beige off-white appearance, suitable for topical formulations.

### Example 19

Silver oxide formulations were prepared according to the general procedure provided in Example 1. The active ingredients were:
silver(I) oxide or silver(II) oxide (0.01%);
zinc oxide (0.02%);
magnesium oxide (0.02%);

The carrier was the standard base described in Example 1. The ratio of whitener, zinc oxide and stabilization agent to silver oxide was 2:1. The ratio of whitener and stabilization agent to zinc oxide was 1:1. As in Example 18, both the silver(I) and the silver(II) oxide based formulations exhibited a generally off-white to slightly beige off-white appearance, suitable for topical formulations.

### Example 20

Anti-microbial activity was evaluated indirectly using a Fisher educational spectrophotometer. This technique uses turbidity as an indicator of microbial growth. Bacterial samples were grown in a Muller-Hinton broth. Upon inoculation with the microbe, 4-5 mg samples of the exemplary formulations were loaded on to a 6 mm sterile disc, dropped into the broth, and allowed to incubate for 24 hours. After a pre-determined time, the samples were introduced to the spectrophotometer and the optical density (OD) measured. The OD reflects the turbidity of a sample, or the relative transparency of a sample to light passing therethrough, to the light detector on the distal side. Increasing OD may be generally correlated with an increased concentration of microbes.

### Example 21

Anti-microbial activity was evaluated using a Bel-Art Colony Counting System, Scienceware Colony Counting System Instrument. The colony counting system may be performed instead of, or complementary to, the above-described spectrophotometric method.

Bacterial samples were inoculated in Muller-Hinton broth and the anti-microbial formulations were loaded, as in the spectrophotometric test described in Example 11. After 24 hours, a drop of the media was taken and streaked on a Muller-Hinton agar plate. After the plates were inoculated for 24 hours, the number of colonies visible in the plates was counted. The number of colonies visible may be generally correlated with decreasing anti-microbial efficacy of the sample formulations.

### Example 22

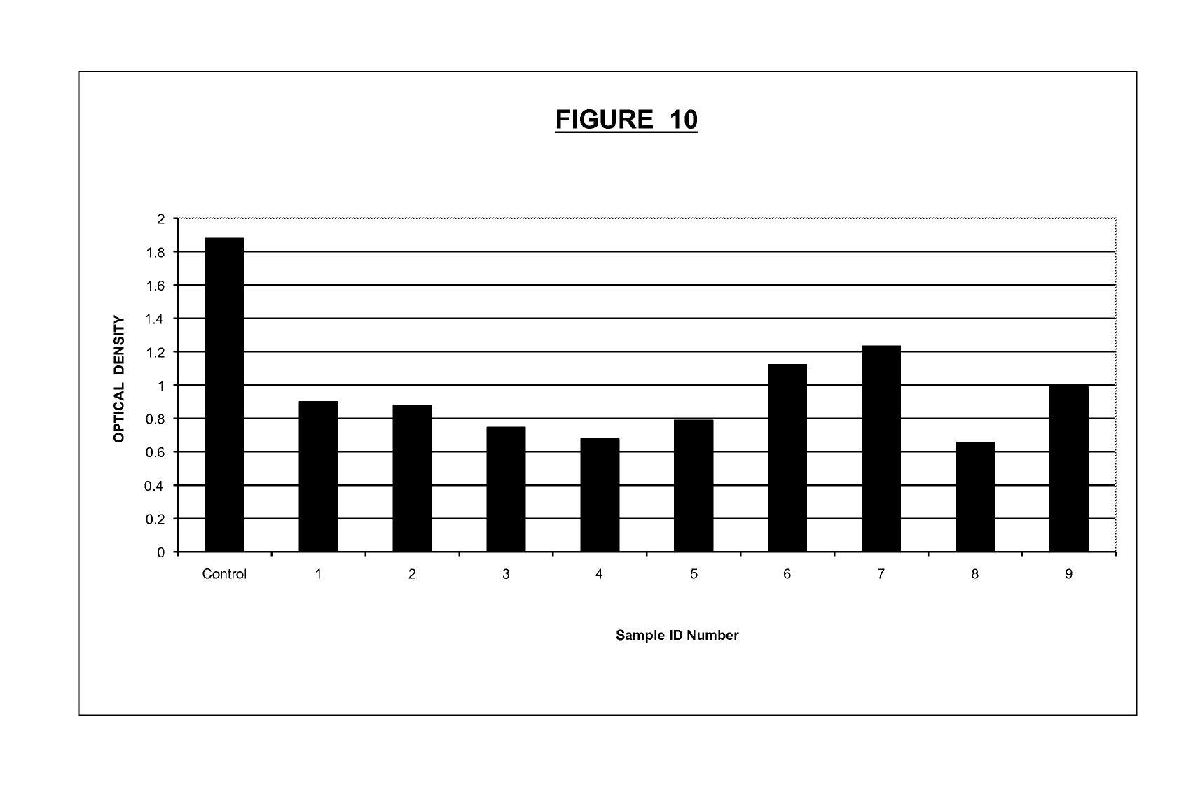

FIG. 10 is a bar graph showing the turbidity of a plurality of cultures, each culture containing a particular anti-microbial formulation. One culture is a control culture, having no anti-microbial components. Sample 1 has no whitener, and no stabilizing agent. Samples 2-9 all contain an inorganic lightener or whitener. Some of these lighteners/whiteners may also act as a stabilizing agent that retards the discoloration process within the formulation.

All of the anti-microbial formulation containing cultures displayed pronounced anti-microbial activity. The least effective anti-microbial formulation was Sample 8, containing 1% Ag₂0 and 7% TiO₂. The most effective anti-microbial formulations were Samples 3-5 and 8, all containing 1% Ag₂O and containing 7% of Ca(OH)₂, Mg(OH)₂, MgCO₃, or MgO, respectively.

TABLE 3

| Sample ID# | Sample Description | Optical Density |
|---|---|---|
| C | Control | 1.878 |
| 1 | AgO (1%) | 0.898 |
| 2 | AgO/CaCO₃ (1%, 7%) | 0.876 |
| 3 | AgO/Ca(OH)₂ (1%, 7%) | 0.745 |
| 4 | AgO/Mg(OH)₂ (1%, 7%) | 0.677 |
| 5 | AgO/MgCO₃ (1%, 7%) | 0.788 |
| 6 | AgO/Bentonite (1%, 7%) | 1.121 |
| 7 | AgO/Ti₂O (1%, 7%) | 1.232 |
| 8 | AgO/MgO (1%, 7%) | 0.656 |
| 9 | AgO/MgSO₄ (1%, 7%) | 0.987 |

### Example 23

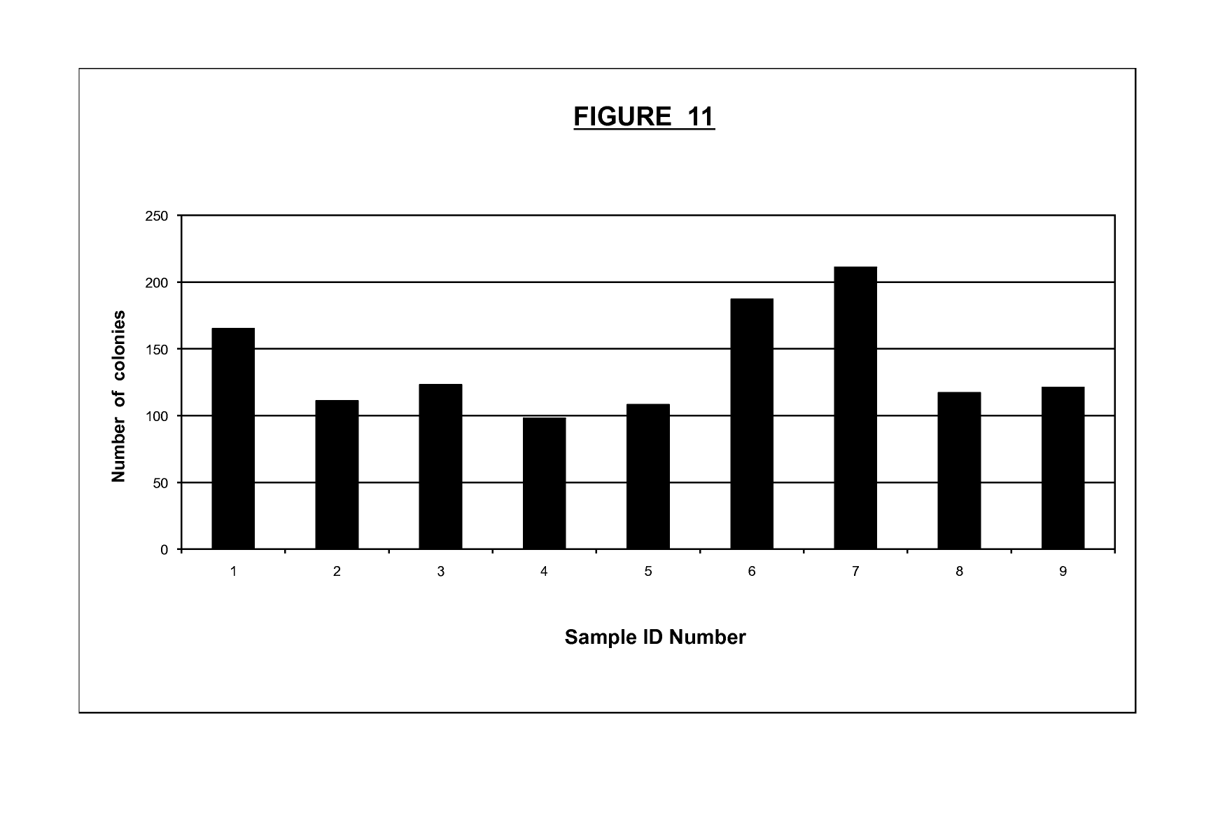

FIG. 11 is a bar graph showing the colony counts for the anti-microbial formulation containing cultures of FIG. 10. The general trend is similar to the turbidity trend observed in Example 18. The most effective anti-microbial formulations were Samples 2-5, 8 and 9, all containing 1% Ag₂O and containing 7% of CaCO₃, Ca(OH)₂, Mg(OH)₂, MgCO₃, MgO, or MgSO₄, respectively. As in Example 20, the least effective anti-microbial formulation was Sample 8, containing 1% Ag₂0 and 7% TiO₂.

TABLE 4

| | | Number of |
|---|---|---|
| Sample ID# | Sample Description | colonies |
| 1 | AgO (1%) | 165 |
| 2 | AgO/CaCO3 (1%, 7%) | 111 |
| 3 | AgO/CaOH2 (1%, 7%) | 123 |
| 4 | AgO/MgOH2 (1%, 7%) | 98 |
| 5 | AgO/MgCO3 (1%, 7%) | 108 |
| 6 | AgO/Bentonite (1%, 7%) | 187 |
| 7 | AgO/Ti2O (1%, 7%) | 211 |
| 8 | AgO/MgO (1%, 7%) | 117 |
| 9 | AgO/MgSO4 (1%, 7%) | 121 |

As used herein in the specification and in the claims section that follows, the term “silver (II) oxide” refers to a silver oxide whose unit structure contains silver and oxygen in a substantially 1:1 molar ratio. The term “silver (II) oxide” is specifically meant to include Ag₄O₄ (often represented as Ag₂O₃.Ag₂O), which may be generally referred to as AgO herein.

As used herein in the specification and in the claims section that follows, the term “silver (I) oxide” refers to a silver oxide whose unit structure contains silver and oxygen in a substantially 2:1 molar ratio. The term “silver (I) oxide” is specifically meant to include Ag₂O.

As used herein in the specification and in the claims section that follows, the terms “homogeneous” and “substantially homogeneous”, with respect to a silver oxide formulation, are meant to be used according to their meaning in the art of topical formulation manufacturing.

As used herein in the specification and in the claims section that follows, the term “whiteness value” with respect to a substance or formulation, refers to a reflectance value, in reflectance units, as determined by a LabScan XE spectrophotometer instrument (HunterLab, VA), or the like. The spectrophotometer must be calibrated whereby the measured reflectance value of the following sample substances, is within 0.40 reflectance units, and preferably within 0.30 reflectance units or 0.20 reflectance units, of the respective measured reflectance values provided below:

| | | | | | | Measured |
|---|---|---|---|---|---|---|
| | Cyan | Magenta | Yellow | Key | Observed | Reflectance |
| Sample ID | (C) | (M) | (Y) | (K) | Color | Units (RU) |
| Sample | 7 | 3 | 2 | 11 | Light gray | 5.21 |
| A1 | | | | | | |
| Sample | 2 | 3 | 4 | 5 | Light gray | 5.67 |
| A2 | | | | | | |
| Sample | 17 | 12 | 20 | 24 | Medium | 4.12 |
| A3 | | | | | gray | |
| Sample | 22 | 14 | 9 | 31 | Medium | 4.55 |
| A4 | | | | | gray | |
| Sample | 16 | 23 | 23 | 69 | Dark gray | 3.49 |
| A5 | | | | | | |

Typically, a light gray formulation exhibits a reflectance of at least about 4.8 RU; a medium gray formulation exhibits a reflectance in a range of about 3.8 to about 4.8 RU; a dark gray formulation exhibits a reflectance of less than about 3.8 RU, and typically between about 1.0 and 3.8 RU or between 2.0 and 3.8 RU.

As used herein in the specification and in the claims section that follows, the term “initial whiteness value” with respect to a substance or formulation, refers to the whiteness value of the substance or formulation, prior to significant exposure to ultraviolet radiation. Typically, the initial whiteness value is measured within several minutes, or within an hour, from the time the substance or formulation is dispensed from its tube, vial, or the like.

As used herein in the specification and in the claims section that follows, the term “gray hue” and the like, with respect to a substance or formulation, is meant to include light gray, medium gray, dark gray, and off-white hues.

As used herein in the specification and in the claims section that follows, the term “stabilization agent” and the like, refers to a substance that retards or otherwise reduces the darkening of silver oxide formulations over time or over exposure to ultraviolet light. The term “stabilization agent” is meant to specifically exclude titania and zinc oxide.

As used herein in the specification and in the claims section that follows, the term “constant exposure to ultraviolet light” and the like relates to conditions identical or substantially identical to those delineated in Example 10, or to conditions determined and demonstrated by an expert in the art to yield identical or highly similar results with respect to the conditions delineated in Example 10.

As used herein in the specification and in the claims section that follows, the term “percent”, or “%”, refers to percent by weight, unless specifically indicated otherwise.

Similarly, the term “ratio”, as used herein in the specification and in the claims section that follows, refers to a weight ratio, unless specifically indicated otherwise.

As used herein in the specification and in the claims section that follows, the term “largely includes”, with respect to a component within a formulation, refers to a weight content of at least 30%.

As used herein in the specification and in the claims section that follows, the term “mainly includes”, with respect to a component within a formulation, refers to a weight content of at least 50%.

As used herein in the specification and in the claims section that follows, the term “predominantly includes”, with respect to a component within a formulation, refers to a weight content of at least at least 65%.

It will be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A topical formulation for application to exposed body tissue, the formulation comprising:
(a) a silver oxide, and
(b) at least one inorganic whitener compound
selected from the group of inorganic whiteners consisting of an inorganic magnesium compound and an inorganic calcium compound,
said silver oxide and said inorganic whitener compound intimately dispersed within a carrier medium,
and wherein a ratio of said inorganic whitener compound, to said silver oxide, is at least 0.2:1, by weight, within the formulation.

2. The topical formulation of claim 1, the formulation containing at least 0.10%, by weight, of said silver oxide.

3. The topical formulation of claim 2, wherein a whiteness value of the formulation is, initially, at least 4 reflective units, and wherein, after constant exposure to said ultraviolet light for 3 days, said value remains at least 3.5 reflective units.

4. The topical formulation of claim 2, wherein first whiteness value of the formulation is at least 4 reflective units, and wherein, after constant exposure to said ultraviolet light for 3 days, a whiteness value of the formulation remains within 1.5 reflective units of said first whiteness value.

5. The topical formulation of claim 1, the formulation containing at least 0.05%, by weight, of said silver oxide.

6. (canceled)

7. The topical formulation of claim 1, wherein said whitener compound is further selected to act as a stabilization agent that partially inhibits a darkening of the formulation when the formulation is exposed to ultraviolet light.

8. The topical formulation of claim 1, wherein the formulation has a light gray hue.

9. The topical formulation of claim 1, wherein said carrier medium includes an oleaginous material.

10. The topical formulation of claim 9, wherein said oleaginous material includes a wax.

11. The topical formulation of claim 9, wherein said oleaginous material includes beeswax.

12. The topical formulation of claim 1, wherein said carrier medium includes a liquid wax ester.

13. The topical formulation of claim 12, wherein said liquid wax ester includes, predominantly includes, or consists essentially of jojoba oil.

14.-15. (canceled)

16. The topical formulation of claim 1, wherein said whitener compound is an inorganic powder.

17. The topical formulation of claim 1, further comprising zinc oxide.

18. The topical formulation of claim 17, wherein said whitener compound is further selected to act as a stabilization agent that partially inhibits a darkening of the formulation when the formulation is exposed to ultraviolet light.

19.-21. (canceled)

22. The topical formulation of claim 1, wherein said silver oxide mainly includes a silver(II) oxide.

23. The topical formulation of claim 1, wherein said silver oxide includes a silver(I) oxide.

24.-28. (canceled)

29. The topical formulation of claim 1, wherein the formulation contains less than 0.5%, less than 0.3%, or less than 0.1% titanium dioxide, or is substantially free of said titanium dioxide.

30.-32. (canceled)

33. The topical formulation of claim 1, wherein said inorganic whitener compound is selected from the group consisting of a magnesium oxide, a magnesium carbonate, a magnesium sulfate, a calcium oxide, a calcium carbonate, and a calcium sulfate.

34.-39. (canceled)

40. A topical formulation for application to exposed body tissue, the formulation comprising:
(a) a silver(II) oxide, and
(b) at least one inorganic whitener compound selected from the group of inorganic whiteners consisting of an inorganic magnesium compound and an inorganic calcium compound,
said silver(II) oxide and said inorganic whitener compound intimately dispersed within a carrier medium,
the formulation containing at least 0.05%, by weight, of said silver(II) oxide,
and wherein a ratio of said inorganic whitener compound, to said silver(II) oxide, is at least 0.2:1, by weight, within the formulation.
